(19) United States
(12) Patent Application Publication
(10) Pub. No.: US 2014/0056897 A1
(43) Pub. Date: Feb. 27, 2014

(54) BISPECIFIC THREE-CHAIN ANTIBODY-LIKE MOLECULES

(71) Applicants: Roland Buelow, Palo Alto, CA (US); Wim van Schooten, Sunnyvale, CA (US)

(72) Inventors: Roland Buelow, Palo Alto, CA (US); Wim van Schooten, Sunnyvale, CA (US)

(73) Assignee: HCO Antibody, Inc., Palo Alto, CA (US)

(21) Appl. No.: 14/001,250

(22) PCT Filed: Mar. 9, 2012
(86) PCT No.: PCT/US12/28607

§ 371 Date: Nov. 11, 2013

Related U.S. Application Data
(60) Provisional application No. 61/469,541, filed on Mar. 30, 2011.
(60) Provisional application No. 61/451,474, filed on Mar. 10, 2011.

(51) Int. Cl. C07K 16/46

(52) U.S. Cl. CPC C07K 16/468
USPC 424/136.1; 530/387.3; 435/69.6

(57) ABSTRACT

The present invention relates to novel bispecific three-chain antigen-binding polypeptides and their preparation and use in the treatment and/or diagnosis of various diseases, and also relates to bispecific three-chain antibody-like molecules (TCAs) capable of activating immune effector cells and their use in diagnosis and/or treatment of various diseases.

### FIELD OF THE INVENTION

The present invention concerns novel bispecific three-chain antigen-binding polypeptides and their preparation and use in the treatment and/or diagnosis of various diseases. The invention particularly relates to bispecific three-chain antibody-like molecules (TCAs) capable of activating immune effector cells and their use in diagnosis and/or treatment of various diseases. The invention specifically concerns bispecific three-chain polypeptides with binding affinity for the CD3 antigen complex, their preparation and use in cancer immunotherapy.

### BACKGROUND OF THE INVENTION

Activation of Immune Effector Cells by Antibodies

The body's immune system serves as a defense against infection, injury and cancer. Two separate but interrelated systems, humoral and cellular immune systems, work together to protect the body. The humoral system is mediated by soluble factors, named antibodies, which neutralize products recognized as being foreign by the body. In contrast, the cellular system involves cells, such as T cells and macrophages, which remove and neutralize foreign invaders

The activation of T cells is critical for the stimulation of immune responses. T cells exhibit immunological specificity and direct most of the cellular immune responses. Although T cells do not secrete antibodies, they are required for the secretion of antibodies by B lymphocytes. T cell activation requires the participation of a number of cell surface molecules, such as the T cell receptor complex, and CD4 or CD8 molecules. The antigen-specific T cell receptor (TcR) is composed of a disulfide-linked heterodimer, membrane glycoprotein with chains, alpha and beta (α and β), or gamma and delta (γ and δ). The TcR is non-covalently linked with a complex of invariant proteins, designated CD3.

The TcR confers antigen specificity and the CD3 structures transduce activation signals to T cells. The CD3 complex contains four subunits. They can contain two zeta subunits, one epsilon subunit and either a gamma or a delta subunit. Antigen binding leads to the cross-linking and activation of the TCR complex. T-cell receptor signaling leads to T-cell activation and IL-2 production and other cytokines in a complex process.

The ligand of the TcR is the MHC-peptide complex on the surface of target cells such as virus-infected cells. After the recognition of the MHC-peptide on the target cell, T cells can have a cytotoxic or an apoptotic effect on the target cell. Especially cytotoxic T cells (CD8 positive T cells) can have advantageous effects by directly removing virus-infected cells. This arm of the cellular immune response is particularly advantageous and is critical for fighting virus infections and eliminating tumor cells.

Activation of the cytotoxic T cell may occur via direct binding of the CD3 antigen without the recognition of the MHC-peptide complex by the TcR. This alternative activation route can be achieved with anti-CD3 antibodies. Non-human monoclonal antibodies have been developed against some of the CD3 chains (subunits), as exemplified by the murine antibodies OKT3, SP34, UCHT1 or 64.1. (See e.g., June, et al., J. Immunol. 136:3945-3952 (1986); Yang, et al., J. Immunol. 137:1097-1100 (1986); and Hayward, et al., Immunol. 64:87-92 (1988)).

Many of these anti-CD3 antibodies bind the epsilon chain which leads to the development of highly activated T cells. Cancer immunotherapy with ordinary monoclonal antibodies does not activate T-lymphocytes because these cells lack the Fcgamma receptor. For that reason bispecific antibodies, one arm recognizing human CD3 and the other a tumor antigen, have a higher cytotoxic potential in in vitro and animal models of cancer. In clinical trials a BiTE antibody was efficacious in non-Hodgkins lymphoma patients at very low doses (Bargou et al., Science 321, p 974-977, 2008). The lowest effective dose was around 0.015 mg/m² per day (milligrams per square meter body surface area per day), several orders of magnitude lower than with ordinary antibodies.

CD3 antibodies are disclosed, for example, in U.S. Pat. Nos. 5,585,097; 5,929,212; 5,968,509; 6,706,265; 6,750,325; 7,381,803; 7,728,114. Bispecific antibodies with CD3 binding specificity are disclosed, for example, in U.S. Pat. Nos. 7,262,276; 7,635,472; and 7,862,813.

Bispecific Antibodies

Bispecific antibodies have shown considerable benefits over monospecific antibodies for the treatment and the detection of cancer. Broad commercial application of bispecific antibodies has been hampered by the lack of efficient/low-cost production methods, the lack of stability of bispecific polypeptides and the lack of long half-lives in humans. A large variety of methods have been developed over the last decades to produce bispecific monoclonal antibodies (BsMAB).

First-generation BsMAbs consists of two heavy and two light chains, one each from two different antibodies. The two Fab regions are directed against two antigens. The Fc region is made up from the two heavy chains and forms the third binding site with the Fc receptor on immune cells, for that reason also called trifunctional antibodies (H. Lindhofer et al., The Journal of Immunology, Vol 155, p 219-225, 1995). Introduction of two different antibodies in one cell line leads to the expression in the supernatant of 10 different IgG molecules consisting of various combinations of heavy and light chains. Therefore, the yield of functional bispecific Ab is low, and purification is often complicated. To overcome this drawback antibodies from different species have been expressed in one cell line which due to the increased incidence of correctly paired Ab facilitates production of BsMAbs. For example, cell lines expressing rat and mouse antibodies secrete functional bispecific Ab because of preferential species-restricted heavy and light chain pairing. Standard methods are used to purify these rat/mouse BsMAb. A rat/mouse hybrid BsMAb (Removab, catumaxomab) has been approved for human use. These non-human(ized) BsMAb products elicit strong immune responses upon repeated administrations and, for that reason, are only indicated for non-chronic use. The mechanism of action of catumaxomab is that one Fab is directed against EpCAM, a tumor antigen, and the other against CD3, a T-lymphocyte antigen. The Fc region additionally binds to a cell that expresses Fc receptors, like a macrophage, a natural killer cell or a dendritic cell. In sum, the tumor cell is connected to one or two cells of the immune system, which subsequently destroy it.

Other types of bispecific antibodies have been designed to overcome certain problems of rat/mouse trifunctional antibodies, such as short half-life, immunogenicity and side-effects caused by cytokine release. They include chemically linked Fabs, consisting only of the Fab regions. Two chemically linked Fab or Fab2 fragments form an artificial antibody that binds to two different antigens, making it a type of bispecific antibody. Antigen-binding fragments (Fab or Fab2) of two different monoclonal antibodies are produced and linked by chemical means like a thioether (Glennie, M J et al., Journal of immunology 139, p 2367-75, 1987). Typically, one of the Fabs binds to a tumor antigen (such as CD30) and the other to a protein on the surface of an immune cell, for example an Fc receptor on a macrophage or CD3 on a T cell. In this way, tumor cells are attached to immune cells, which destroy them. Clinical trials with chemically linked Fabs were conducted for the treatment of cancer which yielded promising results (Peter Borchmann et al., Blood, Vol. 100, No. 9, p 3101-3107, 2002). Because of high production costs this approach was dropped for further development.

Various other methods for the production of multivalent artificial antibodies have been developed by recombinantly fusing variable domains of two antibodies. A single-chain variable fragment (scFv) is a fusion protein of the variable regions of the heavy (VH) and light chains (VL) of immunoglobulins, connected with a short linker peptide of ten to about 25 amino acids. The linker is usually rich in glycine for flexibility, as well as serine or threonine for solubility, and can either connect the N-terminus of the VH with the C-terminus of the VL, or vice versa. Bispecific single-chain variable fragments (di-scFvs, bi-scFvs) can be engineered by linking two scFvs with different specificities. A single peptide chain with two VH and two VL regions is produced, yielding bivalent scFvs. The furthest developed of these are bispecific tandem scFvs, known as bi-specific T-cell engagers (BiTEs). The first BiTEs antibodies bind via one scFv to T cells via the CD3 receptor, and via the other scFv to tumor cells via a tumor specific molecule. For example, Blinatumomab (MT103) is under development for the treatment of non-Hodgkin's lymphoma and acute lymphoblastic leukemia; and is directed towards CD19, a surface molecule expressed on B cells and CD3, a surface molecule expressed on T cells. Similarly, MT110 is under development for the treatment of gastrointestinal and lung cancers; directed towards the EpCAM antigen on tumor cells and CD3. Utilizing the same technology, melanoma (with MCSP specific BiTEs) and acute myeloid leukemia (with CD33 specific BiTEs) are targeted. Another possibility is the creation of bispecific scFvs with linker peptides that are too short for the two variable regions to fold together (about five amino acids), forcing scFvs to dimerize. This type is known as diabodies (Adams et al., British journal of cancer 77, p 1405-12, 1998). Again, these formats can be composed from variable fragments with specificity for two different antigens, in which case they are types of bispecific diabodies. All these technologies lead to proteins with non-human sequences which can lead to immunogenicity after multiple dosings and short half-lives. Bispecific diabodies and BiTES by themselves have short-lives of hours to days. In contrast, natural antibodies have half-lives of weeks.

Another artificial antibody platform is the Dual-Affinity Re-Targeting (DART) platform technology (Macrogenics, Rockville, Md.). This fusion protein technology uses two single-chain variable fragments (scFvs) of different antibodies on a single peptide chain of about 55 kilodaltons.

SCORPION Therapeutics (Emergent Biosolutions, Inc., Seattle, Wash.) is a platform technology combining two antigen-binding domains in a single chain protein. One binding domain is on the C-terminus and a second binding domain on the N-terminus of an effector domain base on immunoglobulin Fc regions.

Tetravalent and bispecific antibody-like proteins are DVD-Igs which are engineered from two monoclonal antibodies (Wu, C. et al., Nature Biotechnology, 25, p 1290-1297, 2007). To construct the DVD-Ig molecule, the V domains of the two mAbs are fused in tandem by a short linker (TVAAP) with the variable domain of the first antibody light (VL) chain at the N terminus, followed by the other antibodies VL and Ck to form the DVD-Ig protein light chain. Similarly, the variable regions of the heavy (VH) chain of the two mAbs are fused in tandem by a short linker (ASTKGP) with the first antibody at the N terminus, followed by the other antibody and the heavy chain constant domains to form the DVD-Ig protein heavy chain (VH1/VL1). All light chain and heavy chain constant domains are preserved in the DVD-Ig design, as they are critical for the formation of a disulfide-linked full IgG-like molecule. Cotransfection of mammalian cells with expression vectors encoding the DVD-Ig light chain and heavy chain leads to the secretion of a single species of an IgG-like molecule with molecular weight of approximately 200 kDa. This molecule has now four binding sites, 2 from each mAb.

### SUMMARY OF THE INVENTION

In one aspect, the invention concerns a bispecific three-chain antibody-like molecule (TCA) comprising

(a) an antibody heavy and light chain pair, or a functional fragment thereof, comprising at antigen-binding region specifically binding to a first binding target and at least one heavy chain constant region sequence; and

(b) a heavy chain antibody comprising an antigen-binding region specifically binding to a second binding target, and a CH2, CH3 and/or CH4 region, in the absence of a CH1 region.

In another aspect, the invention concerns a pharmaceutical composition comprising such bispecific TCA, in admixture with a pharmaceutically acceptable ingredient.

In yet another aspect, the invention concerns a kit comprising a container containing a bispecific TCA of a pharmaceutical composition of the present invention and instructions directing the user to utilize the bispecific TCA or the pharmaceutical composition.

In a further aspect, the invention concerns a method for the production of a bispecific TCA of the present invention comprising expressing the antibody heavy and light chain pair and the heavy chain antibody in a single host cell.

In various embodiments, the host cell may be a prokaryotic or an eukaryotic cell, such as a mammalian cell.

In a still further aspect, the invention concerns a method for the treatment of a cancer, comprising administering to a subject diagnosed with said cancer an effective amount of a bispecific TCA of the present invention.

In various embodiments, the cancer is selected from the group consisting of ovarian cancer, breast cancer, gastrointestinal, brain cancer, head and neck cancer, prostate cancer, colon cancer, lung cancer, leukemia, lymphoma, sarcoma, carcinoma, neural cell tumors, squamous cell carcinomas, germ cell tumors, metastases, undifferentiated tumors, seminomas, melanomas, myelomas, neuroblastomas, mixed cell tumors, and neoplasias caused by infectious agents.

In another aspect, the invention concerns a method for the treatment of an autoimmune disease or inflammatory condition comprising administering to a subject in need an effective amount of the bispecific TCA of the present invention.

In a further aspect, the invention concerns a method for the treatment of an infectious disease caused by bacteria, viruses or parasites, comprising administering to a subject in need an effective amount of a bispecific TCA of the present invention.

In all aspects, the bispecific TCA might be present in various embodiments.

Thus, in one embodiment, in the bispecific TCA the antibody heavy and light chain pair comprises an antigen-binding region specifically binding to a first binding target and a CH1 sequence.

In another embodiment, in the bispecific TCA the antibody heavy and light chain pair comprises an antigen-binding region specifically binding to a first binding target and a CH1 and a CH2 sequence.

In yet another embodiment, in the bispecific TCA the antibody heavy and light chain pair comprises an antigen-binding region specifically binding to a first binding target and a CH1, a CH2, and a CH3 sequence.

In other embodiments, in the bispecific TCA the antibody heavy and light chain pair further comprises a hinge region.

In a further embodiment, in the bispecific TCA the antibody heavy and light chain, or functional fragments thereof, are covalently linked to each other.

In a more particular embodiment, in the bispecific TCA the antibody heavy and light chain, or functional fragments thereof, are linked by a disulfide bond.

In a different embodiment, in the bispecific TCA the heavy chain antibody comprises an antigen-binding region specifically binding to a second binding target and a CH2 region, in the absence of a CH1 region.

In another embodiment, in the bispecific TCA the heavy chain antibody further comprises a CH3 region, in the absence of a CH1 region.

In yet another embodiment, in the bispecific TCA the heavy chain antibody further comprises a CH4 region, in the absence of a CH1 region.

In a further embodiment, the heavy chain antibody further comprises a hinge region.

In further embodiments, the first and second binding targets can be two different antigens, or different epitopes on the same antigen.

In a still further embodiment, the bispecific TCA herein may bind to a cell surface antigen expressed by a target cell and an antigen expressed by an effector cell.

In a particular embodiment, at least one of the first and second binding targets is part of a CD3 complex, such as CD3 epsilon.

In all embodiments, the bispecific TCAs herein may be humanized or human.

### BRIEF DESCRIPTION OF THE FIGURES

FIG. 1. Schematic Diagrams of a human heavy chain polypeptide, a human IgG antibody and a human bispecific 3-chain polypeptide. Co-expression of the heavy chain polypeptide and human antibody in a host cell line yields all three molecules in the supernatant. Purification of individual polypeptides is achieved using standard protein purification technologies such affinity (protein A), size exclusion and hydrophobic interaction chromatography.

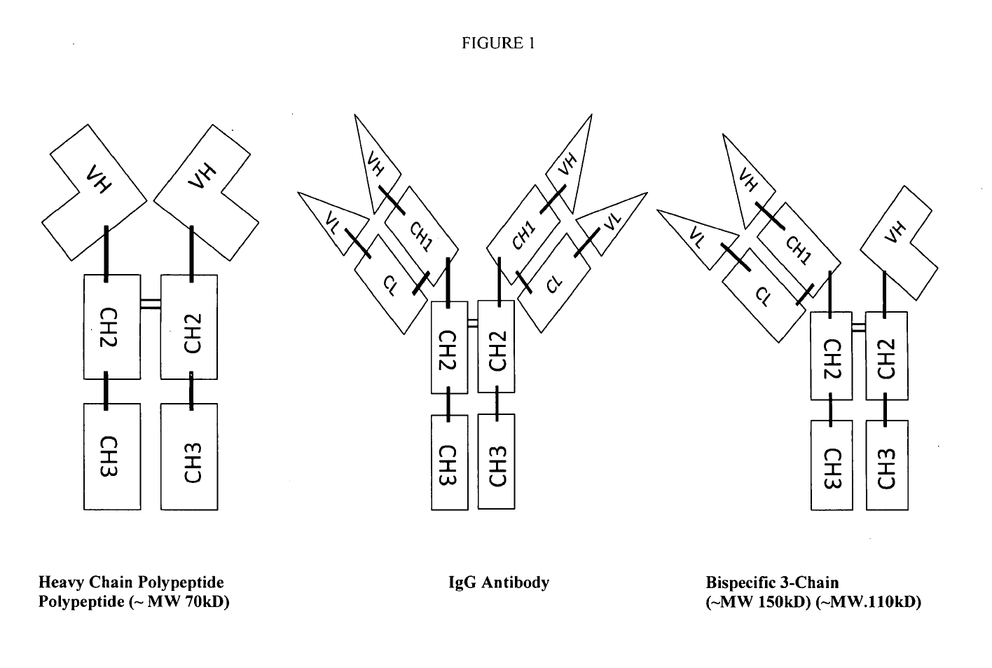

### DETAILED DESCRIPTION OF THE INVENTION

### I. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 2nd. Edition (1989) Cold Spring Harbor Laboratory Press, Cold Spring Harbor, N.Y. Also, for example, Current Protocols in Molecular Biology, Supplement 93, January 2011, John Wiley & Sons, Inc. As appropriate, procedures involving the use of commercially available kits and reagents are generally carried out in accordance with manufacturer defined protocols and/or parameters unless otherwise noted.

It must be noted that as used herein and in the appended claims, the singular forms “a”, “and”, and “the” include plural referents unless the context clearly dictates otherwise.

Throughout this specification and claims, the word “comprise,” or variations such as “comprises” or “comprising,” will be understood to imply the inclusion of a stated integer or group of integers but not the exclusion of any other integer or group of integers.

When trade names are used herein, applicants intend to independently include the trade name product formulation, the generic drug, and the active pharmaceutical ingredient(s) of the trade name product.

All publications and other references mentioned herein are incorporated herein by reference to disclose and describe the methods and/or materials in connection with which the publications are cited. Publications cited herein are cited for their disclosure prior to the filing date of the present application. Nothing here is to be construed as an admission that the inventors are not entitled to antedate the publications by virtue of an earlier priority date or prior date of invention. Further the actual publication dates may be different from those shown and require independent verification.

Unless stated otherwise, the following terms and phrases as used herein are intended to have the following meanings:

The term “antigen” refers to an entity or fragment thereof which can bind to an antibody or trigger a cellular immune response. An immunogen refers to an antigen which can elicit an immune response in an organism, particularly an animal, more particularly a mammal including a human. The term antigen includes regions known as antigenic determinants or epitopes.

As used herein, the term “immunogenic” refers to substances which elicit the production of antibodies, and/or activate T-cells and/or other reactive immune cells directed against an antigen of the immunogen.

An immune response occurs when an individual produces sufficient antibodies, T-cells and other reactive immune cells in response to administered immunogenic compositions.

The term immunogenicity as used herein refers to a measure of the ability of an antigen to elicit an immune response (humoral or cellular) when administered to a recipient. The present invention is concerned with approaches that reduce the immunogenicity of the subject human chimeric or humanized antibodies.

The term “bispecific” is used herein to refer to binding polypeptides that recognize two different antigens. In one embodiment, the “bispecific” polypeptides are three-chain, antigen-binding antibody-like molecules, which recognize two different antigens, by virtue of possessing at least one first antigen combining site specific for a first antigen or hapten, and at least one second antigen combining site specific for a second antigen or hapten. Such polypeptides can be produced by recombinant DNA methods and/or by chemical synthesis. Bispecific polypeptides which have two or more recognition sites for each antigen are specifically included within this definition.

The term “bispecific three-chain antibody like molecule” or “TCA” is used herein to refer to antibody-like molecules comprising, consisting essentially of, or consisting of three polypeptide subunits, two of which comprise, consist essentially of, or consist of one heavy and one light chain of a monoclonal antibody, or functional antigen-binding fragments of such antibody chains, comprising an antigen-binding region and at least one CH domain. This heavy chain/light chain pair has binding specificity for a first antigen. The third polypeptide subunit comprises, consists essentially of, or consists of a heavy chain only antibody comprising an Fc portion comprising CH2 and/or CH3 and/or CH4 domains, in the absence of a CH1 domain, and an antigen binding domain that binds an epitope of a second antigen or a different epitope of the first antigen, where such binding domain is derived from or has sequence identity with the variable region of an antibody heavy or light chain. Parts of such variable region may be encoded by V_{H} and/or V_{L} gene segments, D and J_{H} gene segments, or J_{L} gene segments. The variable region may be encoded by rearranged V_{H}DJ_{H}, V_{L}DJ_{H}, V_{H}J_{L}, or V_{L}J_{L} gene segments.

Antibodies, also referred to as immunoglobulins, generally comprise two identical heavy chains and two identical light chains. Each heavy and light chain comprises an amino terminal domain that is variable and a carboxy terminal end that is constant. The variable domain from one heavy chain (V_{H}) and the variable domain from one light chain (V_{L}) together form an antigen binding site of an antibody. Accordingly, a native antibody generally has two antigen binding sites. Typically, the two heavy chains are covalently bound to each other by disulphide bonds at the constant region (C_{H}), and each heavy chain is covalently bound to the constant region of one of the light chains (C_{L}). The term “antibody” herein is used in the broadest sense and specifically covers monoclonal antibodies, polyclonal antibodies, dimers, multimers, multispecific antibodies (e.g., bispecific antibodies), and antibody fragments, so long as they exhibit the desired biological activity (Miller et al (2003) Jour. of Immunology 170:4854-4861). Antibodies may be murine, human, humanized, chimeric, or derived from other species. An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by CDRs on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule. The immunoglobulins can be derived from any species. In one aspect, however, the immunoglobulin is of human, non-human primate, murine, rat, rabbit or chicken origin. The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al (1991) Sequences of Proteins of Immunological Interest, 5th Ed. Public Health Service, National Institutes of Health, Bethesda, Md.). The constant domains are not involved directly in binding an antibody to an antigen, but exhibit various effector functions, such as participation of the antibody in antibody dependent cellular cytotoxicity (ADCC).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations, which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Köhler et al (1975) Nature 256:495, or may be made by recombinant DNA methods (see for example: U.S. Pat. No. 4,816,567; U.S. Pat. No. 5,807,715). The monoclonal antibodies may also be isolated from phage antibody libraries using the techniques described in Clackson et al (1991) Nature, 352:624-628; Marks et al (1991) J. Mol. Biol., 222:581-597; for example.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies of interest herein include “primatized” antibodies comprising variable domain antigen-binding sequences derived from a non-human primate (e.g., Old World Monkey, Ape etc) and human constant region sequences.

An “intact antibody” herein is one comprising a V_{L} and V_{H} domains, as well as a light chain constant domain (CL) and heavy chain constant domains, CH1, hinge, CH2 and CH3 for secreted IgG. Other isotypes, such as IgM or IgA may have different CH domains. The constant domains may be native sequence constant domains (e.g., human native sequence constant domains) or amino acid sequence variant thereof. The intact antibody may have one or more “effector functions” which refer to those biological activities attributable to the Fc constant region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors.

Depending on the amino acid sequence of the constant domain of their heavy chains, intact antibodies can be assigned to different “classes.” There are five major classes of intact immunoglobulin antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known. Ig forms include hinge-modifications or hingeless forms (Roux et al (1998) J. Immunol. 161:4083-4090; Lund et al (2000) Eur. J. Biochem. 267:7246-7256; US 2005/0048572; US 2004/0229310). The light chains of antibodies from any vertebrate species can be assigned to one of two clearly distinct types, called κ and λ, based on the amino acid sequences of their constant domains.

The term “hypervariable region” when used herein refers to the amino acid residues of an antibody which are responsible for antigen-binding. The hypervariable region generally comprises amino acid residues from a “complementarity determining region” or “CDR” (e.g., residues 24-34 (L1), 50-56 (L2) and 89-97 (L3) in the light chain variable domain and 31-35 (H1), 50-65 (H2) and 95-102 (H3) in the heavy chain variable domain; Kabat et al supra) and/or those residues from a “hypervariable loop” (e.g., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk (1987) J. Mol. Biol., 196:901-917). “Framework Region” or “FR” residues are those variable domain residues other than the hypervariable region residues as herein defined.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment, which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

“Humanized” forms of non-human (e.g., rodent) antibodies, including single chain antibodies, are chimeric antibodies (including single chain antibodies) that contain minimal sequence derived from non-human immunoglobulin. Humanization is a method to transfer the murine antigen binding information to a non-immunogenic human antibody acceptor, and has resulted in many therapeutically useful drugs. The method of humanization generally begins by transferring all six murine complementarity determining regions (CDRs) onto a human antibody framework (Jones et al, (1986) Nature 321:522-525). These CDR-grafted antibodies generally do not retain their original affinity for antigen binding, and in fact, affinity is often severely impaired. Besides the CDRs, select non-human antibody framework residues must also be incorporated to maintain proper CDR conformation (Chothia et al (1989) Nature 342:877). The transfer of key mouse framework residues to the human acceptor in order to support the structural conformation of the grafted CDRs has been shown to restore antigen binding and affinity (Riechmann et al (1992) J. Mol. Biol. 224, 487-499; Foote and Winter, (1992) J. Mol. Biol. 224:487-499; Presta et al (1993) J. Immunol. 151, 2623-2632; Werther et al (1996) J. Immunol. Methods 157:4986-4995; and Presta et al (2001) Thromb. Haemost. 85:379-389). For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see U.S. Pat. Nos. 5,225,539; 6,548,640; 6,982,321; 5,585,089; 5,693,761; 6,407,213; Jones et al (1986) Nature, 321:522-525; and Riechmann et al (1988) Nature 332:323-329.

A “functional Fc region” possesses an “effector function” of a native-sequence Fc region. Exemplary “effector functions” include C1q binding; CDC; Fc-receptor binding; ADCC; phagocytosis; down-regulation of cell-surface receptors (e.g., B-cell receptor), etc. Such effector functions generally require the Fc region to be combined with a binding domain (e.g. an antibody-variable domain) and can be assessed using various assays as disclosed, for example, in definitions herein.

A “native-sequence Fc region” comprises an amino acid sequence identical to the amino acid sequence of an Fc region found in nature. Native-sequence human Fc regions include a native-sequence human IgG1 Fc region (non-A and A allotypes); native-sequence human IgG2 Fc region; native-sequence human IgG3 Fc region; and native-sequence human IgG4 Fc region, as well as naturally occurring variants thereof.

A “variant Fc region” comprises an amino acid sequence that differs from that of a native-sequence Fc region by virtue of at least one amino acid modification, preferably one or more amino acid substitution(s). Preferably, the variant Fc region has at least one amino acid substitution compared to a native-sequence Fc region or to the Fc region of a parent polypeptide, e.g., from about one to about ten amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and most preferably at least about 90% homology therewith, more preferably at least about 95% homology therewith.

“Homology” between two sequences is determined by sequence identity. If two sequences, which are to be compared with each other, differ in length, sequence identity preferably relates to the percentage of the nucleotide residues of the shorter sequence which are identical with the nucleotide residues of the longer sequence. Sequence identity can be determined conventionally with the use of computer programs such as the Bestfit program (Wisconsin Sequence Analysis Package, Version 8 for Unix, Genetics Computer Group, University Research Park, 575 Science Drive Madison, Wis. 53711). Bestfit utilizes the local homology algorithm of Smith and Waterman, Advances in Applied Mathematics 2 (1981), 482-489, in order to find the segment having the highest sequence identity between two sequences. When using Bestfit or another sequence alignment program to determine whether a particular sequence has for instance 95% identity with a reference sequence of the present invention, the parameters are preferably so adjusted that the percentage of identity is calculated over the entire length of the reference sequence and that homology gaps of up to 5% of the total number of the nucleotides in the reference sequence are permitted. When using Bestfit, the so-called optional parameters are preferably left at their preset (“default”) values. The deviations appearing in the comparison between a given sequence and the above-described sequences of the invention may be caused for instance by addition, deletion, substitution, insertion or recombination. Such a sequence comparison can preferably also be carried out with the program “fasta20u66” (version 2.0u66, September 1998 by William R. Pearson and the University of Virginia; see also W. R. Pearson (1990), Methods in Enzymology 183, 63-98, appended examples and http://workbench.sdsc.edu/). For this purpose, the “default” parameter settings may be used.

The term “Fc-region-comprising antibody” refers to an antibody that comprises an Fc region. The C-terminal lysine (residue 447 according to the EU numbering system) of the Fc region may be removed, for example, during purification of the antibody or by recombinant engineering the nucleic acid encoding the antibody. Accordingly, an antibody having an Fc region according to this invention can comprise an antibody with or without K447.

The term “single chain antibody” as used herein means a single polypeptide chain containing one or more antigen binding domains that bind an epitope of an antigen, where such domains are derived from or have sequence identity with the variable region of an antibody heavy or light chain. Parts of such variable region may be encoded by V_{H} or V_{L} gene segments, D and J_{H} gene segments, or J_{L} gene segments. The variable region may be encoded by rearranged V_{H}DJ_{H}, V_{L}DJ_{H}, V_{H}J_{L}, or V_{L}J_{L} gene segments. V-, D- and J-gene segments may be derived from humans and various animals including birds, fish, sharks, mammals, rodents, non-human primates, camels, lamas, rabbits and the like.

The term “heavy chain only antibody” or “heavy chain antibody” or “heavy chain polypeptide” as used herein means a single chain antibody comprising heavy chain CH2 and/or CH3 and/or CH4 but no CH1 domain. In one embodiment, the heavy chain antibody is composed of an antigen-binding domain, at least part of a hinge region and CH2 and CH3 domains. In another embodiment, the heavy chain antibody is composed of an antigen-binding domain, at least part of a hinge region and a CH2 domain. In a further embodiment, the heavy chain antibody is composed of an antigen-binding domain, at least part of a hinge region and a CH3 domain. Heavy chain antibodies in which the CH2 and/or CH3 domain is truncated are also included herein. In a further embodiment the heavy chain is composed of an antigen binding domain, and at least one CH (CH1, CH2, CH3, or CH4) domain but no hinge region. The heavy chain only antibody can be in the form of a dimer, in which two heavy chains are disulfide bonded other otherwise, covalently or non-covalently attached with each other. The heavy chain antibody may belong to the IgG subclass, but antibodies belonging to other subclasses, such as IgM, IgA, IgD and IgE subclass, are also included herein. In a particular embodiment, the heavy chain antibody is of the IgG1, IgG2, IgG3, or IgG4 subtype, in particular IgG1 subtype.

Heavy chain antibodies constitute about one fourth of the IgG antibodies produced by the camelids, e.g. camels and llamas (Hamers-Casterman C., et al. Nature. 363, 446-448 (1993)). These antibodies are formed by two heavy chains but are devoid of light chains. As a consequence, the variable antigen binding part is referred to as the VHH domain and it represents the smallest naturally occurring, intact, antigen-binding site, being only around 120 amino acids in length (Desmyter, A., et al. J. Biol. Chem. 276, 26285-26290 (2001)). Heavy chain antibodies with a high specificity and affinity can be generated against a variety of antigens through immunization (van der Linden, R. H., et al. Biochim. Biophys. Acta. 1431, 37-46 (1999)) and the VHH portion can be readily cloned and expressed in yeast (Frenken, L. G. J., et al. J. Biotechnol. 78, 11-21 (2000)). Their levels of expression, solubility and stability are significantly higher than those of classical F(ab) or Fv fragments (Ghahroudi, M. A. et al. FEBS Lett. 414, 521-526 (1997)). Sharks have also been shown to have a single VH-like domain in their antibodies termed VNAR. (Nuttall et al. Eur. J. Biochem. 270, 3543-3554 (2003); Nuttall et al. Function and Bioinformatics 55, 187-197 (2004); Dooley et al., Molecular Immunology 40, 25-33 (2003)).

An antibody and binding molecules, including the heavy chain only antibodies and bispecific three-chain antibody-like molecules (TCAs) herein, that “specifically binds to” or is “specific for” a particular polypeptide or an epitope on a particular polypeptide is one that binds to that particular polypeptide or epitope on a particular polypeptide without substantially binding to any other polypeptide or polypeptide epitope.

An antibody or binding molecule, including the heavy chain only antibodies and bispecific three-chain antibody-like molecules (TCAs) herein, “which binds” an antigen of interest, is one that binds the antigen with sufficient affinity such that the antibody or binding molecule is useful as a diagnostic and/or therapeutic agent in targeting the antigen, and does not significantly cross-react with other proteins. In such embodiments, the extent of binding of the antibody or other binding molecule to a non-targeted antigen will be no more than 10% as determined by fluorescence activated cell sorting (FACS) analysis or radioimmunoprecipitation (RIA).

“Complement dependent cytotoxicity” and “CDC” refer to the lysing of a target in the presence of complement. The complement activation pathway is initiated by the binding of the first component of the complement system (C1q) to a molecule (e.g. an antibody) complexed with a cognate antigen.

“Binding affinity” generally refers to the strength of the sum total of noncovalent interactions between a single binding site of a molecule (e.g., an antibody or other binding molecule) and its binding partner (e.g., an antigen or receptor). The affinity of a molecule X for its partner Y can generally be represented by the dissociation constant (Kd). Affinity can be measured by common methods known in the art, including those described herein. Low-affinity antibodies bind antigen (or receptor) weakly and tend to dissociate readily, whereas high-affinity antibodies bind antigen (or receptor) more tightly and remain bound longer.

A “functional” or “biologically active” antibody or binding molecule (including heavy chain only antibodies and bispecific three-chain antibody-like molecules (TCAs) herein) is one capable of exerting one or more of its natural activities in structural, regulatory, biochemical or biophysical events. For example, a functional antibody or other binding molecule, e.g. TCA, may have the ability to specifically bind an antigen and the binding may in turn elicit or alter a cellular or molecular event such as signaling transduction or enzymatic activity. A functional antibody or other binding molecule, e.g. TCA, may also block ligand activation of a receptor or act as an agonist or antagonist. The capability of an antibody or other binding molecule, e.g. TCA, to exert one or more of its natural activities depends on several factors, including proper folding and assembly of the polypeptide chains.

The term “vector,” as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operably linked. Such vectors are referred to herein as “recombinant expression vectors” (or simply, “recombinant vectors”). In general, expression vectors of utility in recombinant DNA techniques are often in the form of plasmids. In the present specification, “plasmid” and “vector” may be used interchangeably as the plasmid is the most commonly used form of vector.

The term “host cell” (or “recombinant host cell”), as used herein, is intended to refer to a cell that has been genetically altered, or is capable of being genetically altered by introduction of an exogenous polynucleotide, such as a recombinant plasmid or vector. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

“Mammal” for purposes of treatment refers to any animal classified as a mammal, including humans, non-human primates, domestic and farm animals, and zoo, sports, or pet animals, such as dogs, horses, cats, cows, etc. Specifically included within the definition are rodents, such as mice and rats and animals creating antibody diversity by gene conversion.

### II. Modes for Carrying Out the Invention

1. Bispecific Three-Chain Antibody-Like Molecules (TCAs)

The present invention discloses novel bispecific antibody like molecules (binding polypeptides), which find utility, for example, in the treatment and/or diagnosis of human diseases. The novel bispecific antibody like molecules consist of three polypeptide chains and are called three chain antibodies (TCAs). Two of such polypeptide chains comprise at least the portion of an antibody heavy and light chain that is required to form an antigen-binding domain, and at least one antibody heavy chain constant region sequence, i.e. a CH1 and/or CH2 and/or CH3 and/or CH4 region sequence. In certain embodiments, the heavy chain sequence may also include a hinge region. In one embodiment, the two polypeptide chains are one heavy and one light chain of a monoclonal antibody specifically binding to a first antigen.

The third polypeptide chain is a heavy chain only antibody comprising an Fc portion comprising CH2 and/or CH3 and/or CH4 domains, in the absence of a CH1 domain, and an antigen binding domain that binds an epitope of a second antigen, where such binding domain is derived from or has sequence identity with the variable region of an antibody heavy or light chain. Parts of such variable region may be encoded by V_{H} or V_{L} gene segments, D and J_{H} gene segments, or J_{L} gene segments. The variable region may be encoded by rearranged V_{H}DJ_{H}, V_{L}DJ_{H}, V_{H}J_{L}, or V_{L}J_{L} gene segments. V-, D- and J-gene segments may be derived from humans and various animals including, without limitation, birds, fish. The first and second antigens are different from each other, i.e. the TCA is bispecific.

In certain embodiments, the CH regions can be truncated, provided the remaining sequence is sufficient to retain the function of the full-length CH region.

Although bispecific TCAs can be prepared by chemical synthesis, they are typically produced by methods of recombinant DNA technology, such as co-expression of the three chains making up the molecule in a single recombinant host cell, or co-expression of a heavy chain polypeptide and an antibody, e.g. a human antibody. In addition, the antibody heavy and light chains can also be expressed using a single polycistronic expression vector. The antibody component of the TCA can also be produced by phage display. Co-expression of the heavy chain polypeptide and antibody in a single host cell yields three molecules (antibody, heavy chain polypeptide, and TCA) in the supernatant. Purification of individual polypeptides is achieved using standard protein purification technologies such as affinity (protein A) chromatography, size exclusion chromatography and/or hydrophobic interaction chromatography. TCAs are sufficiently different in size and hydrophobicity that purification can be performed using standard procedures.

The amount of antibody and heavy chain polypeptide produced in a single host cell can be minimized through engineering of constant regions of the antibody and the heavy chain such that homodimerization is favored over heterodimerization, e.g. by introducing self-complementary interactions (see e.g. WO 98/50431 for possibilities, such as “protuberance-into-cavity” strategies (see WO 96/27011)). It is therefore another aspect of the present invention to provide a method for producing a TCA in a recombinant host, the method including the step of: expressing in a recombinant host cell a nucleic acid sequences encoding at least an antibody and a heavy chain polypeptide, wherein said antibody and said heavy chain polypeptide differ in their constant regions sufficiently to reduce or prevent homodimer formation but increase TCA formation.

TCAs without any-non human amino acid sequences may be produced. Such TCAs are non-immunogenic and stable molecules with long half-lives similar to natural antibodies in humans.

Compared to traditional monoclonal antibodies the potency of TCAs is increased.

In one embodiment, the present invention concerns TCAs that bind to two cell surface antigens.

The invention specifically concerns TCAs binding to human CD3. For example, heavy chain only antibody polypeptide may be combined with a heavy and light chain, or a functional fragment thereof, comprising at least an antigen-binding domain and at least one of CH1, CH2, CH3 and CH4 domains, from a monoclonal antibody. The heavy chain only antibody may be specific for human CD3 while the monoclonal antibody (mAb) portion of the TCA may be specific for target cells, including cancer cells, such as cells of ovarian, breast, gastrointestinal, brain, head and neck, prostate, colon, and lung cancers, and the like, as well as hematologic tumors such as B-cell tumors, including leukemias, lymphomas, sarcomas, carcinomas, neural cell tumors, squamous cell carcinomas, germ cell tumors, metastases, undifferentiated tumors, seminomas, melanomas, myelomas, neuroblastomas, mixed cell tumors, neoplasias caused by infectious agents, and other malignancies, cells infected with a pathogen, autoreactive cells causing inflammation and/or autoimmunity. In certain embodiments, the TCAs will have binding specificity for CD3 and tumor antigens, such as, for example, the HER-2/Neu receptor, other growth factor receptors such as EGFR, HER3, HER4, VEGFR1 and VEGFR2 receptor, B-cell markers such as CD19, CD20, CD22, CD37, CD72, etc, T-cell markers such as CD25 or CD11b, other leukocyte cell surface markers such as CD33 or HLA-DR, etc, cytokines such as TNF, interleukins, receptors for these cytokines such as members of the TNF receptor family, and the like.

In other embodiments, the bispecific TCAs herein may have binding specificity for proteins expressed by pathogens, such as viruses, bacteria or parasites. In further embodiment, the bispecific TCAs specifically bind virus infected cells or viral proteins expressed on the surface of infected cells or viral particles. In further embodiments, the bispecific TCAs specifically bind to parasite proteins expressed on the surface of cells with intracellular parasites.

Exemplary anti-CD3 monoclonal antibodies that can be included in the bispecific TCA's of the present invention include, without limitation, OKT3 (Otho) and its variants, including aglycosylated variants. Preferably, the antibodies are fully human antibodies. Generally, the anti-CD3 antibody has one or more of the following characteristics: the antibody binds to CD3 positive (CD3+) cells but not CD3 negative (CD3−) cells; the anti-CD3 antibody induces antigenic modulation which involves alteration (e.g., decrease) of the cell surface expression level or activity of CD3 or the T cell receptor (TcR).

For example, the CD3 specific heavy chain antibody may be combined with heavy and light chain of a mAb such as Rituxan® (specific for CD20 on B cells, including B cell tumors), Avastin® (bevacizumab, an anti-VEGF antibody), Herceptin® (trastuzumab, an anti-HER2 antibody), etc. The CD3 specific single chain peptide can also be combined with antibodies specific for other tumor antigens such as PMSA (Prostate Membrane Specific Antigen), etc. CD3 specific singe chain peptides can also be paired with antibodies recognizing Influenza virus, HIV, Dengue virus, or other virus infected cells.

The invention also discloses TCAs binding to a cell surface antigen and a soluble antigen.

The invention also concerns TCAs that bind to two soluble antigens or two different epitopes on one antigen, such as one soluble antigen. Thus, for example, TCAs binding to two different epitopes on the HER2 antigen or on CD3 are specifically included herein.

TCAs binding to two soluble antigens or two epitopes of one soluble antigen may be able to crosslink such antigens. In animals or humans administration of such TCAs may result in clearance of the target antigens from circulation.

2. Recombinant Production of TCAs

As discussed above, The TCAs herein are typically produced by methods of recombinant DNA technology, such as co-expression of the three chains making up the molecule in a single recombinant host cell.

For recombinant production of the TCAs herein, the nucleic acid encoding the three chains is isolated and inserted into a replicable vector for further cloning (amplification of the DNA) or for expression. DNA encoding the desired single chain antibodies is readily isolated and sequenced using conventional procedures (e.g., by using oligonucleotide probes that are capable of binding specifically to genes encoding the heavy and light chains of the antibody variant). Many vectors are available. The vector components generally include, but are not limited to, one or more of the following: a signal sequence, an origin of replication, one or more marker genes, an enhancer element, a promoter, and a transcription termination sequence.

The heterologous signal sequence selected preferably is one that is recognized and processed (i.e., cleaved by a signal peptidase) by the host cell. For prokaryotic host cells that do not recognize and process the native antibody signal sequence, the signal sequence is substituted by a prokaryotic signal sequence selected, for example, from the group of the alkaline phosphatase, penicillinase, lpp, or heat-stable enterotoxin II leaders. For yeast secretion the native signal sequence may be substituted by, e.g., the yeast invertase leader, .alpha. factor leader (including Saccharomyces and Kluyveromyces α-factor leaders), or acid phosphatase leader, the C. Albicans glucoamylase leader, or the signal described in WO 90/13646. In mammalian cell expression, mammalian signal sequences as well as viral secretory leaders, for example, the herpes simplex gD signal, are available.

The DNA for such precursor region is ligated in reading frame to DNA encoding the antibody.

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the antibody nucleic acid, such as DHFR, thymidine kinase, metallothionein-I and -II, preferably primate metallothionein genes, adenosine deaminase, ornithine decarboxylase, etc.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity.

Alternatively, host cells (particularly wild-type hosts that contain endogenous DHFR) transformed or co-transformed with DNA sequences encoding antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

A suitable selection gene for use in yeast is the trp1 gene present in the yeast plasmid YRp7 (Stinchcomb et al., Nature, 282:39 (1979)). The trp1 gene provides a selection marker for a mutant strain of yeast lacking the ability to grow in tryptophan, for example, ATCC No. 44076 or PEP4-1. Jones, Genetics, 85:12 (1977). The presence of the trp1 lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene.

In addition, vectors derived from the 1.6 μm circular plasmid pKD1 can be used for transformation of Kluyveromyces yeasts. Alternatively, an expression system for large-scale production of recombinant calf chymosin was reported for K. lactis. Van den Berg, Bio/Technology, 8:135 (1990). Stable multi-copy expression vectors for secretion of mature recombinant human serum albumin by industrial strains of Kluyveromyces have also been disclosed. Fleer et al., Bio/Technology, 9:968-975 (1991).

Expression and cloning vectors usually contain a promoter that is recognized by the host organism and is operably linked to the antibody nucleic acid. Promoters suitable for use with prokaryotic hosts include the phoA promoter, beta-lactamase and lactose promoter systems, alkaline phosphatase, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding the antibody.

Promoter sequences are known for eukaryotes. Virtually all eukaryotic genes have an AT-rich region located approximately 25 to bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoter sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phospho-fructokinase, glucose-6-phosphate isomerase, 3-phosphoglycerate mutase, pyruvate kinase, triosephosphate isomerase, phosphoglucose isomerase, and glucokinase. Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

Antibody transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. Alternatively, the rous sarcoma virus long terminal repeat can be used as the promoter.

Transcription of DNA encoding the antibodies of this invention by higher eukaryotes is often increased by inserting an enhancer sequence into the vector. Many enhancer sequences are now known from mammalian genes (globin, elastase, albumin, alpha-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the antibody-encoding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the antibody. One useful transcription termination component is the bovine growth hormone polyadenylation region. See WO94/11026 and the expression vector disclosed therein.

Polycistronic expression vectors, as described, for example, in U.S. Pat. No. 4,713,339, can also be used to express the subunits of the bispecific TCAs herein. In the polycistronic expression vector, the coding sequences of the subunits may be separated by appropriate cleavage sites.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published Apr. 12, 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234), Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

TCAs synthesized in plants can be produced in a variety of ways. Since the first report of antibody production in N. tabacum plants (Hiatt et al., 1989, Nature, 342:76-78), antibodies have been expressed in moss (for review, see Decker and Reski, 2008, Bioprocess Biosyst. Eng., 31, 3-9), algae (for review, see Franklin and Mayfield, 2005, Expert Opin. Biol. Ther., 5, 225-235) and various dicot and monocot species, such as tobaco, rice. For review see, for example, De Muynck et al., 2010, Plant Biotechnology Journal, 8(5):529-563. Transgenic plants or plant cells producing antibodies have also been described (Hiatt et al., 1989, Nature, 342:76-78), and useful plants for this purpose include corn, maize, tobacco, soybean, alfalfa, rice, and the like. Constitutive promoters that can for instance be used in plant cells are the CaMV 35S and 19S promoters, Agrobacterium promoters nos and ocs. Other useful promoters are light inducible promoters such as rbcS. Tissue-specific promoters can for instance be seed-specific, such as promoters from zein, napin, betaphaseolin, ubiquitin, or tuber-specific, leaf-specific (e.g. useful in tobacco), root-specific, and the like. It is also possible to transform the plastid organelle by homologous recombination, to express proteins in plants. Methods and means for expression of proteins in recombinant plants or parts thereof, or recombinant plant cell culture, are known to the person skilled in the art and have been for instance been described in (Giddings et aI, 2000; WO 01/64929; WO 97/42313; U.S. Pat. Nos. 5,888,789, 6,080,560; See for practical guidelines: Methods In Molecular Biology vol. 49 “Plant Gene Transfer And Expression Protocols”, Jones H, 1995).

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC cells; FS4 cells; and a human hepatoma line (Hep G2).

Host cells are transformed with the above-described expression or cloning vectors for antibody production and cultured in conventional nutrient media modified as appropriate for inducing promoters, selecting transformants, or amplifying the genes encoding the desired sequences.

Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. No. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

In a preferred embodiment, the host cell according to the method of the invention is capable of high-level expression of human immunoglobulin, i.e. at least 1 pg/cell/day, preferably at least 10 pg/cell/day and even more preferably at least 20 pg/cell/day or more without the need for amplification of the nucleic acid molecules encoding the single chains in said host cell.

Preferably, host cells according to the invention contain in their genome between 1 and 10 copies of each recombinant nucleic acid to be expressed. In the art, amplification of the copy number of the nucleic acid sequences encoding a protein of interest in e.g. CHO cells can be used to increase expression levels of the recombinant protein by the cells (see e.g. Bendig M. M. (1988) Genet. Eng. 7:91-127; Cockett et al, 1990, Bio/technology 8:662-667; and U.S. Pat. No. 4,399,216). This is currently a widely used method. However, a significant time-consuming effort is required before a clone with a desired high copy number and high expression levels has been established, and moreover clones harboring very high copy numbers (up to hundreds) of the expression cassette often are unstable (e.g. Kim et al., 1998, Biotechnol. Bioeng. 58:73-84). It is therefore a preferred embodiment of the present invention to use host cells that do not require such amplification strategies for high-level expression of the bispecific TCAs of interest. This allows fast generation of stable clones of host cells that express the mixture of single chain antibodies according to the invention in a consistent manner. We provide evidence that host cells according to the invention can be obtained, subcloned and further propagated for at least around 30 cell divisions (population doublings) while expressing the mixture of single chain antibodies according to the invention in a stable manner, in the absence of selection pressure. Therefore, in certain aspects the methods of the invention include culturing the cells for at least 20, preferably 25, more preferably 30 population doublings, and in other aspects the host cells according to the invention have undergone at least 20, preferably 25, more preferably 30 population doublings and are still capable of expressing the TCAs according to the present invention.

The TCAs expressed by the cells according to the invention may be recovered from the cells or preferably from the cell culture medium, by methods generally known to persons skilled in the art. Such methods may include one or more of precipitation, centrifugation, filtration, viral filtration, size-exclusion chromatography, affinity chromatography, cation- and/or anion-exchange chromatography, hydrophobic interaction chromatography, and the like.

3. Pharmaceutical Composition

It is another aspect of the present invention to provide pharmaceutical compositions comprising one or more TCAs of the present invention in admixture with a suitable pharmaceutically acceptable carrier. Pharmaceutically acceptable carriers as used herein are exemplified, but not limited to, adjuvants, solid carriers, water, buffers, or other carriers used in the art to hold therapeutic components, or combinations thereof.

Therapeutic formulations of the TCAs used in accordance with the present invention are prepared for storage by mixing bispecific TCA having the desired degree of purity with optional pharmaceutically acceptable carriers, excipients or stabilizers (see, e.g. Remington's Pharmaceutical Sciences 16th edition, Osol, A. Ed. (1980)), such as in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactide, degradable lactic acid-glycolic acid copolymers, and poly-D-(−)-3-hydroxybutyric acid.

Anti-CD3 antibody formulations are disclosed, for example, in U.S. Patent Publication No. 20070065437, the entire disclosure is expressly incorporated by reference herein. Similar formulations can be used for the bispecific TCAs of the present invention. The main components of such formulations are a pH buffering agent effective in the range of 3.0 to 6.2, a salt, a surfactant, and an effective amount of a TCA with anti-CD3 specificity.

4 Treatment and Diagnosis

It is another aspect of the present invention to provide TCAs for use in the treatment or diagnosis of a human or animal subject. Methods to treat human subjects, including but not limited to cancer patients, with the bispecific TCAs herein are specifically within the scope of the present invention. In another aspect, the invention provides the use of TCAs for the preparation of a medicament for use in the treatment or diagnosis of a disease or disorder in a human or animal subject. In certain embodiments, the disease or condition is a tumor, such as cancer, such as, for example, ovarian cancer, breast cancer, gastrointestinal, brain cancer, head and neck cancer, prostate cancer, colon cancer, lung cancer, hematologic tumors such as B-cell tumors, including leukemias, lymphomas, sarcomas, carcinomas, neural cell tumors, squamous cell carcinomas, germ cell tumors, metastases, undifferentiated tumors, seminomas, melanomas, myelomas, neuroblastomas, mixed cell tumors, neoplasias caused by infectious agents, and other malignancies.

In addition to cancer immunotherapy, the bispecific TCAs of the present invention find utility, for example, in the treatment of various autoimmune diseases and/or inflammatory conditions, including transplant rejection and Type I diabetes, or infectious diseases caused by bacteria, viruses or parasites.

Autoimmune diseases include, for example, Acquired Immunodeficiency Syndrome (AIDS, which is a viral disease with an autoimmune component), alopecia areata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, crest syndrome, Crohn's disease, Degos' disease, dermatomyositis-juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus (Type I diabetes), juvenile chronic arthritis (Still's disease), juvenile rheumatoid arthritis, Meniere's disease, mixed connective tissue disease, multiple sclerosis, myasthenia gravis, pemacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjogren's syndrome, stiff-man syndrome, systemic lupus erythematosus, Takayasu arteritis, temporal arteritis/giant cell arteritis, ulcerative colitis, uveitis, vitiligo and Wegener's granulomatosis.

Inflammatory disorders, include, for example, chronic and acute inflammatory disorders. Examples of inflammatory disorders include Alzheimer's disease, asthma, atopic allergy, allergy, atherosclerosis, bronchial asthma, eczema, glomerulonephritis, graft vs. host disease, hemolytic anemias, osteoarthritis, sepsis, stroke, transplantation of tissue and organs, vasculitis, diabetic retinopathy and ventilator induced lung injury.

Examples of infectious diseases include, but are not limited to, diseases caused by viruses, such as Human immunodeficiency virus (HIV); influenza virus (INV); encephalomyocarditis virus (EMCV), stomatitis virus (VSV), parainfluenza virus; rhinovirus; hepatitis A virus; hepatitis B virus; hepatitis C virus; apthovirus; coxsackievirus; Rubella virus; rotavirus; Dengue virus; yellow fever virus; Japanese encephalitis virus; infectious bronchitis virus; Porcine transmissible gastroenteric virus; respiratory syncytial virus; papillomavirus; Herpes simplex virus; varicellovirus; Cytomegalovirus; variolavirus; Vacciniavirus; suipoxvirus and coronavirus.

Further examples of infectious diseases include, but are not limited to, diseases caused by microbes such as Actinobacillus actinomycetemcomitans; Bacille Calmette-Gurin; Blastomyces dermatitidis; Bordetella pertussis; Campylobacter consisus; Campylobacter recta; Candida albicans; Capnocytophaga sp.; Chlamydia trachomatis; Eikenella corrodens; Entamoeba histolitica; Enterococcus sp.; Escherichia coli; Eubacterium sp.; Haemophilus influenzae; Lactobacillus acidophilus; Leishmania sp.; Listeria monocytogenes; Mycobacterium vaccae; Neisseria gonorrhoeae; Neisseria meningitidis; Nocardia sp.; Pasteurella multocida; Plasmodium falciparum; Porphyromonas gingivalis; Prevotella intermedia; Pseudomonas aeruginosa; Rothia dentocarius; Salmonella typhi; Salmonella typhimurium; Serratia marcescens; Shigella dysenteriae; Streptococcus mutants; Streptococcus pneumoniae; Streptococcus pyogenes; Treponema denticola; Trypanosoma cruzi; Vibrio cholera; and Yersinia enterocolitica.

Further details of the invention are illustrated by the following non-limiting examples.

### Example 1

### Generation of Genetically Engineered Rats Expressing Heavy Chain-Only Antibodies

Construction of Modified Human Ig Loci on YACs and BACs.

A human IgH locus was constructed and assembled in several parts, which involved the modification and joining of rat C region genes, which were then joined downstream of human V_{H}6-D-J_{H} region. Two BACs with separate clusters of human V_{H} genes [BAC3 and BAC6] were then co-injected with a BAC encoding the assembled (human V_{H}6-D-J_{H}-rat C) fragment.

For the rat constant region three BACs were identified [N12, M5 and I8]. These were individually shaved, while a 170 bp homology arm matching the 5′ end of shaved M5 was added to the 3′ end of shaved N12 and a 100 bp homology arm matching the 5′ end of shaved 18 was added to the 3′ end of shaved M5. These modified BACs when put together contain a large part of the rat constant (C) region including E (enhancer) μ, s (switch) μ, Cμ, Cδ, sγ2b, Cγ2b, sε, Cε, sα, Cα and 3′E. The CH1 regions of rat Cμ and rat Cγ2b located in shaved N12 and I8, respectively, were removed. The modified N12 and M5 were then joined to yield the BAC N12M5. These BAC modifications were carried out using the Red®/ET Recombineering technology.

As multiple BAC modifications in E. coli frequently deleted repetitive regions such as switch sequences and enhancers, technologies were developed to assemble sequences with overlapping ends in S. cerevisiae as circular YAC (cYAC) and, subsequently, to convert such a cYAC into a BAC. Advantages of YACs include their large size, the ease of homologous alterations in the yeast host and the sequence stability, whilst BACs propagated in E. coli offer the advantages of easy preparation and large yield. Additionally, detailed restriction mapping and sequencing analysis can be better achieved in BACs than in YACs. Two self-replicating S. cerevisiae/E. Coli shuttle vectors, pBelo-CEN-URA, and pBelo-CEN-HYG were constructed. Briefly, S. cerevisiae CEN4 was cut out as an AvrII fragment from pYAC-RC (Marchuk and Collins, 1988) and ligated to SpeI-linearised pAP599. The resulting plasmid contains CEN4 cloned between URA3 and Hyg^{R}. From this plasmid, an ApaLI-BamHI fragment containing URA3 followed by CEN4 or, a Pm1I-SphI fragment containing Hyg^{R} followed by CEN4, was cut out, and ligated to ApaLI and BamHI or HpaI and SphI digested pBACBelo11 (New England Biolabs) to yield pBelo-CEN-URA and pBelo-CEN-HYG.

Restriction analysis of the modified 18 revealed that the sγ2b region in this BAC was 2.5 to 3 kb shorter than expected. To assemble the ˜125 kb rat C region lacking CH1 in Cμ as well as Cγ2b (N12M5I8) and maintain its authentic configuration, equal moles of the following purified fragments were mixed: 51 kb SwaI-NotI from modified N12M5, 6.5 kb XbaI fragment encompassing the authentic sγ2b region to replace the shortened sγ2b in the modified 18 (previously cloned into pBeloBAC11), 81 kb NruI fragment from the modified I8, and the PCR-amplified pBelo-CEN-URA containing homology arms (65 bp) at either end corresponding to the sequence immediately downstream of rat JH in N12 and the 3′ end of the rat C region in I8 (primers 321 and 322). The DNA mix, in which each fragment overlaps from 65 bp to 15 kb with its neighbouring fragment at both the 5′ and 3′ end, was transformed into S. cerevisiae AB1380 cells using the standard spheroplast transformation procedure to select for URA⁺ clones (Nelson and Brownstein, 1994). Through homologous recombination in yeast associated with the transformation, a cYAC containing N12M5I8 in expected configuration was assembled. The overlapping junctions of the neighbouring fragments in the resulting cYAC were confirmed by PCR analysis using yeast genomic DNA as template. After purification the cYAC was transformed into E. coli DH10 competent cells (Invitrogen) via electroporation. The correct BAC N12M5I8 was identified by extensive restriction mappings and sequencing.

BAC1 was modified in 3 steps to yield BAC 1-Shaved containing the human V_{H}6-D-J_{H} region. Firstly, BAC1 was partially digested by PvuI and re-ligated to remove the sequence upstream of human V_{H}6-1. Secondly, the resulting shortened BAC 1 was digested at a PacI site immediately downstream of the human J_{H}s as well as an AscI site in the vector backbone to remove a 41 kb fragment. Subsequently, a 2.5 kb fragment located immediately downstream of the rat J_{H}s was amplified from rat genomic DNA and flanked by Pad and AscI sites (primers 140 and 141). This PacI-AscI fragment which provides the overlap to the 5′ end of modified N12 was ligated with Pad and AscI double digested shortened BAC1 to yield BAC1-Shaved.

Subsequently, BAC 3-1N12M5I8 was constructed via the cYAC/BAC strategy. This BAC contains the following regions from 5′ to 3′: the 11.3 kb sequence from the 3′ end of BAC3 (providing the overlap to BAC3 when co-injected into the rat genome), the entire BAC1-Shaved followed by the entire N12M5I8. Conveniently, the 3′ end of BAC3 overlaps 5.5 kb with the 5′ end of BAC1-Shaved. To assemble 3-1N12M5I8, the 11.3 kb BAC3 fragment was amplified by PCR, and then mixed with the PvuI-AscI fragment from BAC1-Shaved, the MluI fragment encompassing the entire N12M518, and the amplified pBelo-CEN-URA with homology arms at both ends corresponding to the 5′ end of the 11.3 kb BAC3 fragment and the 3′ end of 18. This DNA mix was used to transform AB1380 cells. Correct joining of each fragment in the transformation was confirmed by PCR analysis. After converting the assembled 3-1N12M5I8 region into a BAC, it was thoroughly checked by restriction mapping. The heavy chain gene region in BAC 3-1N12M5I8 can be cut out entirely together with the S. cerevisiae URA3 gene at its 3′ end as a NotI fragment. When integrated into the rat genome, the existence of the URA3 in this large DNA fragment facilitates the identification of the transgene.

Finally, a 10.6 kb fragment located at the 5′ end of human V_{H} loci in BAC3 was amplified using primers 411, 412 and integrated into BAC6 to provide overlap to BAC3. This modified BAC was named BAC6(+3). The 3′ of the human V_{H} loci in BAC6 contains highly repetitive sequences which renders the manipulation via recombination very difficult in this region. Hence, we chose to integrate the BAC3 fragment into the vector backbone of BAC6. To achieve this, the pBelo-CEN+URA vector with 65 bp homology arms added to either end which overlaps with the 3′ end of the 10.6 kb BAC3 fragment as well as the 5′ end of human V_{H} loci in BAC6, respectively, was amplified using primers 427 and 414. The DNA mix including equal moles of the 10.6 kb BAC3 fragment, the amplified pBelo-CEN+URA, and uncut BAC6 was transformed into AB1380 S. cerevisiae spheroplasts, and URA⁺ transformants were selected. PCR analysis were used to identify the correct integrants that contain the BAC3 fragment followed by pBelo-BAC+URA located between the vector backbone of BAC6 and the 5′ end of BAC6 human V_{H} loci. After converting this cYAC into a BAC, it was thoroughly checked by restriction mapping. Digesting BAC6(+3) with AscI releases a fragment approximately 220 kb containing the entire BAC6 human V_{H} loci and at its 3′ end, the 10.6 kb overlapping BAC3 fragment.

DNA Purification

Linear YACs, circular YACs and BAC fragments after digests, were purified by electro-elution using Elutrap™ (Schleicher and Schuell) (Gu et al., 1992) from strips cut from 0.8% agarose gels run conventionally or from pulsed-field-gel electrophoresis (PFGE). The DNA concentration was usually several ng/μl in a volume of ˜100 μl. For fragments up to ˜200 kb the DNA was precipitated and re-dissolved in micro-injection buffer (10 mM Tris-HCl pH 7.5, 100 mM EDTA pH 8 and 100 mM NaCl but without Spermine/Spermidine) to the desired concentration.

The purification of circular YACs from yeast was carried out using Nucleobond AX silica-based anion-exchange resin (Macherey-Nagel, Germany). Briefly, spheroplasts were made using zymolyase or lyticase and pelleted (Davies et al., 1996). The cells then underwent alkaline lysis, binding to AX100 column and elution as described in the Nucleobond method for a low-copy plasmid. Contaminating yeast chromosomal DNA was hydrolyzed using Plasmid-Safe™ ATP-Dependent DNase (Epicentre Biotechnologies) followed by a final cleanup step using SureClean (Bioline). An aliquot of DH10 electrocompetent cells (Invitrogen) was then transformed with the circular YAC to obtain BAC colonies. For the separation of the insert DNA for microinjection, 150-200 kb, from BAC vector DNA, ˜10 kb, a filtration step with sepharose 4B-CL was used (Yang et al., 1997).

Gel Analyses

Purified YAC and BAC DNA was analysed by restriction digest and separation on conventional 0.7% agarose gels (Sambrook and Russell, 2001). Larger fragments, 50-200 kb, were separated by PFGE (Biorad Chef Mapper™) at 8° C., using 0.8% PFC Agaraose in 0.5% TBE, at 2-20 sec switch time for 16 h, 6V/cm, 10 mA. Purification allowed a direct comparison of the resulting fragments with the predicted size obtained from the sequence analysis. Alterations were analysed by PCR and sequencing.

Microinjection

Outbred SD/Hsd strain animals were housed in standard microisolator cages under approved animal care protocols in animal facility that is accredited by the Association for the Assessment and Accreditation for Laboratory Animal Care (AAALAC). The rats were maintained on a 14-10 h light/dark cycle with ad libitum access to food and water. Four to five week old SD/Hsd female rats were injected with 20-25 IU PMSG (Sigma-Aldrich) followed 48 hours later with 20-25 IU hCG (Sigma-Aldrich) before breeding to outbred SD/Hsd males. Fertilized 1-cell stage embryos were collected for subsequent microinjection. Manipulated embryos were transferred to pseudopregnant SD/Hsd female rats to be carried to parturition.

Purified DNA encoding recombinant immunoglobulin loci was resuspended in microinjection buffer with 10 mM Spermine and 10 mM Spemidine. The DNA was injected into fertilized oocytes at various concentrations from 0.5 to 3 ng/μl.

Plasmid DNA or mRNA encoding ZFNs specific for rat immunoglobulin genes were injected into fertilized oocytes at various concentrations from 0.5 to 10 ng/ul.

Zinc-Finger Nucleases (ZFNs)

ZFNs specific for rat immunoglobulin genes were generated.

The ZFN specific for rat Ckappa had the following binding site:

(SEQ ID NO: 1)
ATGAGCAGCACCCTCtcgttgACCAAGGCTGACTATGAA

ZFNs specific for rat J-locus sequences had the following binding sites:

(SEQ ID NO: 2)
CAGGTGTGCCCATCCagctgaGTTAAGGTGGAG
and
(SEQ ID NO: 3)
CAGGACCAGGACACCTGCAgcagcTGGCAGGAAGCAGGT

Rats with Transloci.

Transgenic rats carrying artificial heavy chain immunoglobulin loci in unrearranged configuration were generated. The included constant region genes encode IgM, IgD, IgG2b, IgE, IgA and 3′ enhancer. RT-PCR and serum analysis (ELISA) of transgenic rats revealed productive rearrangement of transgenic immunoglobulin loci and expression of heavy chain only antibodies of various isotypes in serum. Immunization of transgenic rats resulted in production of high affinity antigen-specific heavy chain only antibodies.

Novel Zinc-Finger-Nuclease Knock-Out Technology.

For further optimization of heavy chain-only antibody generation in transgenic rats, knockout rats with inactivated endogenous rat immunoglobulin loci were generated.

For the inactivation of rat heavy immunoglobulin heavy chain expression and rat □ light chain expression, ZFNs were microinjected into single cell rat embryos. Subsequently, embryos were transferred to pseudopregnant female rats and carried to parturition. Animals with mutated heavy chain and light chain loci were identified by PCR. Analysis of such animals demonstrated inactivation of rat immunoglobulin heavy and light chain expression in mutant animals.

### Example 2

### Generation of Antigen-Specific Heavy Chain-Only Antibodies in Rats

For the generation of antigen-specific heavy chain-only antibodies in rats, genetically engineered rats expressing heavy chain only antibodies are immunized in various ways.

Immunization with Inactivated Virus

Influenza viruses with various different hemagglutinin and neuraminidase genes is provided by the Immunology and Pathogenesis Branch, Influenza Division, CDC, Atlanta, Ga. Virus stock is propagated in the allantoic cavities of 10-day-old embryonated chicken eggs and purified through a 10%-50% sucrose gradient by means of ultracentrifugation. Viruses are resuspended in phosphate-buffered saline and inactivated by treatment with 0.05% formalin at 4° C. for 2 weeks. Inactivated virus and alumn solution (Pierce) are mixed in a 3:1 ratio and incubated at room temperature for 1 h before immunization. Genetically engineered rats expressing heavy chain-only antibodies are immunized with whole inactive.

Immunization with Proteins or Peptides

Typically immunogens (proteins or peptides) are diluted to 0.05-0.15 ml with sterile saline and combined with adjuvant to a final volume of 0.1-0.3 ml. Many appropriate adjuvants are available (i.e. heat inactivated Bordetella pertussis, aluminium hydroxide gel, Quil A or saponin, bacterial lipopolysaccharide or anti-CD40) but none have the activity of Complete Freund's Adjuvant (CFA) and Incomplete Freund's Adjuvant (IFA). The concentration of soluble immunigens such as proteins and peptides may vary between 5 μg and 5 mg in the final preparation. The first immunization (priming) with immunogen in CFA is administered intraperitoneally and/or subcutaneously and/or intramuscularly. If intact cells are used as immunogens they are best injected intraperitoneally and/or intraveneously. Cells are diluted in saline and 1 to 20 million cells are administered per injection. Cells that survive in the rat will yield best immunization results. After the first immunization with immunogens in CFA a second immunization in IFA (booster) is usually delivered 4 weeks later. This sequence leads to the development of B cells producing high affinity antibodies. If the immunogen is weak booster immunizations are administered every 2 weeks until a strong humoral response is achieved. The immunogen concentrations can be lower in booster immunizations and intravenous routes can be used. Serum is collected from rats every 2 weeks to determine the humoral response.

For the generation of anti-human CD3e antibodies genetically engineered rats are immunized with his-tagged, recombinant human CD3e (Creative Biomart, Shirley, N.Y.).

Immunization with Cells

Human Jurkat cells are grown in tissue culture. Expression of human CD3e is analyzed by incubation of Jurkat cells with monoclonal antibody OKT3. Subsequently, unbound OKT3 is removed by washing of the cells, and bound OKT3 is detected with anti-mouse IgG conjugated with fluoresceine and flow cytometric analysis.

Rat T cell hybridoma cells are transfected by electroporation with an eukaryotic expression plasmid encoding human CD3 as described (Transy et al., 1989). Transfectants expressing human CD3 are enriched by FACS and propagated in tissue culture.

Genetically engineered rats expressing HCO antibodies, are immunized by injection of 30×10(6) Jurkat cells intraperitoneally. Four and eight weeks after the primary immunization rats are immunized with rat T cells expressing human CD3. Animals expressing anti-human CD3e heavy chain only antibodies are used for the isolation of monoclonal heavy chain only anti-CD3e antibodies

DNA-Based Immunization Protocols

Gene vaccines, or the use of antigen-encoding DNAs to immunize, represent an alternative approach to the development of strong antibody responses in rats.

The route of DNA inoculation is in general the skin, muscle and any other route that supports transfection and expression of the antigen. Purified plasmid DNAs that have been designed to express antigens such an influenza virus hemagglutinin glycoprotein or other human or viral antigens are used. Routes of DNA inoculation include the following: intravenous (tail vein), intraperitoneal; intramuscular (both quadriceps), intranasal, intradermal (such as foot pad), and subcutaneous (such as scruff of the neck). In general, 10-100 μg of DNA is administered in 100 μl of saline per inoculation site or DNA is administered with appropriate vehicles such as gold particles or certain formulations (http://www.incellart.com/index.php?page=genetic-immunization&menu=3.3) that facilitate uptake and transfection of cells. The immunization scheme is similar to the protocol described above; primary immunization followed by booster immunizations.

Purification of Heavy Chain Only Antibodies

For the purification of antibodies, blood is collected from immunized rats and serum or plasma is obtained by centrifugation, which separates the coagulated cell pellet from the liquid top phase containing serum antibodies. Antibodies from serum of plasma are purified by standard procedures. Such procedures include precipitation, ion exchange chromatography, and/or affinity chromatography. For the purification of IgG protein A or protein G can be used (Bruggemann et al., JI, 142, 3145, 1989).

### Example 3

### Isolation of Antibody Expressing B Cells from Rats

Isolation of B Cells from Spleen, Lymph Nodes or Peripheral Blood

A single-cell suspension is prepared from the spleen or lymph nodes of an immunized rat. Cells can be used without further enrichment, after removal of erythrocytes or after the isolation of B cells, memory B cells, antigen-specific B cells or plasma cells. Enrichment can lead to better results and as a minimum removal of erythrocytes is recommended. Memory B cells are isolated by depletion of unwanted cells and subsequent positive selection. Unwanted cells, for example, T cells, NK cells, monocytes, dendritic cells, granulocytes, platelets, and erythroid cells are depleted using a cocktail of antibodies against CD2, CD14, CD16, CD23, CD36, CD43, and CD235a (Glycophorin A). Positive selection with antibodies specific for IgG or CD19 results in highly enriched B cells (between 50%-95%). Antigen-specific B cells are obtained by exposing cells to antigen(s) tagged with fluorescent markers and/or magnetic beads. Subsequently, cells tagged with fluorochrome and/or magnetic beads are separated using (flow cytometry or a fluorescence activated cell sorter [FACS]) a FACS sorter and/or magnets. As plasma cells may express little surface Ig, intracellular staining may be applied. IgM positive B cell memory cells are isolated using antibodies specific for IgM and CD27.

Isolation of B Cells by Fluorescence Activated Cell Sorting

FACS-based methods are used to separate cells by their individual properties. It is important that cells are in a single-cell suspension. Single cell suspensions prepared from peripheral blood, spleen or other immune organs of immunized rats are mixed with fluorochrome-tagged antibodies specific for B cell markers such as CD19, CD138, and CD27. Alternatively, cells are incubated with fluorochrome-tagged antigens. The cell concentration is between 1-20 million cells/ml in an appropriate buffer such as PBS. For example, memory B cells cells can be isolated by selecting cells positive for CD27 and negative for CD45R. Plasma cells can be isolated by selecting for cells positive for CD138 and negative for CD45R. Cells are loaded onto the FACS machine and gated cells are deposited into 96 well plates or tubes containing media. If necessary positive controls for each fluorochrome are used in the experiment, which allows background subtraction to calculate the compensation.

Isolation of B Cells from Bone Marrow

Bone marrow plasma cells (BMPCs) are isolated from immunized animals as described (Reddy et al., 2010). Muscle and fat tissue are removed from the harvested tibias and femurs. The ends of both tibias and femurs are clipped with surgical scissors and bone marrow is flushed out with a 26-gauge insulin syringe (Becton Dickinson, BD). Bone marrow is collected in sterile-filtered buffer no. 1 (PBS, 0.1% BSA, 2 mM EDTA). Bone marrow cells are collected by filtration through a cell strainer (BD) with mechanical disruption and washed with 20 ml PBS and collected in a 50 ml tube (Falcon, BD). Bone marrow cells are centrifuged at 335 g for 10 min at 4° C. Supernatant is decanted and the cell pellet is resuspended in 3 ml of red cell lysis buffer (eBioscience) and shaken gently at 25° C. for 5 min. Cell suspension is diluted with 20 ml of PBS and centrifuged at 335 g for 10 min at 4° C. Supernatant is decanted and cell pellet resuspended in 1 ml of buffer no. 1

Bone marrow cell suspensions are incubated with biotinylated anti-CD45R and anti-CD49b antibodies. The cell suspension is then rotated at 4° C. for 20 min. This is followed by centrifugation at 930 g for 6 min at 4° C., removal of supernatant and re-suspension of the cell pellet in 1.5 ml of buffer no. 1. Streptavidin conjugated M28 magnetic beads (Invitrogen) are washed and resuspended according to the manufacturer's protocol. Magnetic beads (50 ul) are added to each cell suspension and the mixture is rotated at 4° C. for 20 min. The cell suspensions are then placed on Dynabead magnets (Invitrogen) and supernatant (negative fraction, cells unconjugated to beads) are collected and cells bound to beads are discarded.

Prewashed streptavidin M280 magnetic beads are incubated for 30 min at 4° C. with biotinylated anti-CD138 with 0.75 ug antibody per 25 ul of magnetic beads mixture. Beads are then washed according to the manufacturer's protocol and resuspended in buffer no. 1. The negative cell fraction (depleted of CD45R+ and CD49b+ cells) collected as above is incubated with 50 ul of CD138-conjugated magnetic beads and the suspension is rotated at 4° C. for 30 min. Beads with CD138+ bound cells are isolated by the magnet, washed 3 times with buffer no. 1, and the negative (CD138−) cells unbound to beads are discarded. The positive CD138+ bead-bound cells are collected and stored at 4° C. until further processed.

### Example 4

### Generation of Hybridomas

Isolated B cells are immortalized by fusion with myeloma cells such as X63 or YB2/0 cells as described (Köhler and Milstein, Nature, 256, 495, 1975). Hybridoma cells are cultured in selective media and antibody producing hybridoma cells are generated by limiting dilution or single cell sorting.

### Example 5

### Isolation of cDNAs Encoding Heavy Chain Only Antibodies

Generation of cDNA Sequences from Isolated Cells

Isolated cells are centrifuged at 930 g at 4° C. for 5 min. Cells are lysed with TRI reagent and total RNA is isolated according to the manufacturer's protocol in the Ribopure RNA isolation kit (Ambion). mRNA is isolated from total RNA with oligo dT resin and the Poly(A) purist kit (Ambion) according to the manufacturer's protocol. mRNA concentration is measured with an ND-1000 spectrophotometer (Nanodrop).

The isolated mRNA is used for first-strand cDNA synthesis by reverse transcription with the Maloney murine leukemia virus reverse transcriptase (MMLV-RT, Ambion). cDNA synthesis is performed by RT-PCR priming using 50 ng of mRNA template and oligo dT primers according to the manufacturer's protocol of Retroscript (Ambion). After cDNA construction, PCR amplification is performed to amplify heavy chain only antibodies. A list of primers is shown in Table 1:

TABLE 1

| Human VH Gene | Oligo Sequence | VH Gene Matches |
|---|---|---|
| VH1 | ATGGACTGGACCTGGAGGATCC | 1-02, 1-03, 1-08, 7-04.1 |
| VH1-24 | ATGGACTGCACCTGGAGGATCC | 1-24 |
| VH2 | TCCACGCTCCTGCTGCTGAC | 2-05 |
| VH2-26 | GCTACACACTCCTGCTGCTGACC | 2-26 |
| VH3 | ATGGAGTTTGGGCTGAGCTGG | 3-11, 3-23, 3-30, 3-33 |
| VH3-07 | ATGGAATTGGGGCTGAGCTG | 3-07 |
| VH3-09 | ATGGAGTTGGGACTGAGCTGGA | 3-09 |
| VH3-35 | ATGGAATTTGGCCTGAGCTGG | 3-35 |
| VH3-38 | ATGCAGTTTGTGCTGAGCTGG | 3-38 |
| VH4 | TGAAACACCTGTGGTTCTTCC | 4-04, 4-28, 4-31, 4-34 |
| VH4-39 | TGAAGCACCTGTGGTTCTTCC | 4-39 |
| VH6 | TCATCTTCCTGCCCGTGCTGG | 6-01 |
| Rat IgM CH2 | GCTTTCAGTGATGGTCAGTGTGCTTATGAC | |

A 50 ul PCR reaction consists of 0.2 mM forward and reverse primer mixes, 5 ul of Thermopol buffer (NEB), 2 ul of unpurified cDNA, 1 ul of Taq DNA polymerase (NEB) and 39 ul of double-distilled H₂O. The PCR thermocycle program is 92° C. for 3 min; 4 cycles (92° C. for 1 min, 50° C. for 1 min, 72° C. for 1 min); 4 cycles (92° C. for 1 min, 55° C. for 1 min, 72° C. for 1 min), 20 cycles (92° C. for 1 min, 63° C. for 1 min, 72° C. for 1 min); 72° C. for 7 min, 4° C. storage. PCR gene products are gel purified and DNA sequenced.

### Example 6

### Cloning and Expression of Recombinant Heavy Chain-Only Antibodies

PCR products are subcloned into a plasmid vector. For expression in eukaryotic cells cDNA encoding heavy chain only antibody are cloned into an expression vector as described (Tiller et al., 2008).

Alternatively, the genes encoding heavy chain only antibodies are cloned into a minicircle producing plasmid as described (Kay et al., 2010).

Alternatively, genes encoding heavy chain only antibodies are synthesized from overlapping oligonucleotides using a modified thermodynamically balanced inside-out nucleation PCR (Gao at al., 2003) and cloned into an eukaryotic expression vector.

Alternatively, genes encoding heavy chain-only antibodies are synthesized and cloned into a plasmid.

For the assembly of multiple expression cassettes encoding various heavy chain only antibodies in an artificial chromosome, multiple expression cassettes are ligated with each other and subsequently cloned into a BAC vector, which is propagated in bacteria. For transfection ElectroMAX™ DH10B™ cells from Invitrogen are used (http://tools.invitrogen.com/content/sfs/manuals/18290015.pdf). Alternatively, ligated expression cassettes are further ligated with yeast artificial chromosome arms, which are propagated in yeast cells (Davies et al., 1996).

Plasmid Purification

GenElute™ plasmid miniprep kits from Sigma-Aldrich are used for plasmid isolation from ˜5 ml (or larger) overnight bacterial culture (http://www.sigmaaldrich.com/life-science/molecular-biology/dna-and-rna-purification/plasmid-miniprep-kit.html). This involves harvesting bacterial cells by centrifugation followed by alkaline lysis. DNA is then column-bound, washed and eluted and ready for digests or sequencing.

BAC Purification

NucleoBond^{R} BAC100 from Clontech is a kit designed for BAC purification (http://www.clontech.com/products/detail.asp?tabno=2&product id=186802). For this bacteria are harvested from 200 ml culture and lysed by using a modified alkaline/SDS procedure. The bacterial lysate is cleared by filtration and loaded onto the equilibrated column, where plasmid DNA binds to the anion exchange resin. After subsequent washing steps, the purified plasmid DNA is eluted in a high-salt buffer and precipitated with isopropanol. The plasmid DNA is reconstituted in TE buffer for further use.

YAC Purification

Linear YACs, circular YACs and BAC fragments after digests, are purified by electro-elution using Elutrap™ (Schleicher and Schuell) (Gu et al., 1992) from strips cut from 0.8% agarose gels run conventionally or from pulsed-field-gel electrophoresis (PFGE). The purified DNA is precipitated and re-dissolved in buffer to the desired concentration.

The purification of circular YACs from yeast is carried out using Nucleobond AX silica-based anion-exchange resin (Macherey-Nagel, Germany). Briefly, spheroplasts are made using zymolyase or lyticase and pelleted (Davies et al., 1996). The cells then undergo alkaline lysis, binding to AX100 column and elution as described in the Nucleobond method for a low-copy plasmid. Contaminating yeast chromosomal DNA is hydrolyzed using Plamid-Safe™ ATP-Dependent DNase (Epicentre Biotechnologies) followed by a final cleanup step using SureClean (Bioline). An aliquot of DH10 electrocompetent cells (Invitrogen) is then transformed with the circular YAC to obtain BAC colonies (see above). For the separation of the insert DNA, 150-200 kb, from BAC vector DNA, ˜10 kb, a filtration step with sepharose 4B-CL is used (Yang et al., 1997).

Transfection of Cells with Plasmid or BAC DNA

For the expression of recombinant heavy chain only antibodies, eukaryotic cells are transfected as described (Andreason and Evans, 1989; Baker and Cotton, 1997; http://www.millipore.com/cellbiology/cb3/mammaliancell). Cells expressing heavy chain only antibodies are isolated using various selection methods. Limiting dilution or cell sorting is used for the isolation of single cells. Clones are analyzed for heavy chain only antibody expression.

### Example 7

### Production of Single Heavy Chain Polypeptides Reactive with CD3-Epsilon

HCOA rats are immunized as described in U.S. Pat. No. 7,728,114 B2.

Rat spleenocytes are fused with myeloma cells and B-cell hybridomas are screened for the expression of anti-human CD3e antibodies.

Alternatively, rat B cells are isolated and cultured as described (J Immunol Methods 1993, 160(1):117-127). Culture supernatants are screened for the presence of anti-human CD3e antibodies and cDNAs encoding such antibodies are generated by RT-PCR from isolated RNA.

### Example 8

### Production of Bispecific TCAs

CHO cells are transfected with three expression plasmids encoding an antibody heavy chain, an antibody light chain, and an anti-CD3 heavy chain only antibody. Following selection of transfectants expressing TCA, cells are further propagated in tissue culture medium. TCA is purified from the culture supernatant by protein A chromatography, followed by ion exchange chromatography and/or size exclusion chromatography.

The composition of purified recombinant heavy chain only antibodies is analyzed by analytical cation exchange chromatography as well as mass spectrometry based techniques as described (Persson et al 2010).

### REFERENCES FOR THE EXAMPLES

Popov, A. V., Bützler, C., Frippiat, J.-P., Lefranc, M. P., and Brüggemann, M. (1996) Assembly and extension of yeast artificial chromosomes to build up a large locus. Gene, 177, 195-205
Popov, A. V., Zou, X., Xian, J., Nicholson, I. C. and Brüggemann, M. (1999) A human immunoglobulin □ locus is similarly well expressed in mice and humans. J. Exp. Med., 189, 1611-1619
Marchuk. D. and Collins, F. S. (1988) pYAC-RC, a yeast artificial chromosome vector for cloning DNA cut with infrequently cutting restriction endonucleases. Nucl. Acids Res., 16, 7743.
Ma, B., Pan, S. J., Zupancic, M. L. and Cormack, B. P. (2007) Assimilation of NAD⁺ precursors in Candida glabrata. Mol. Microbiol., 66, 14-25.
Gietz D and Woods R A (1998) Transformation of yeast by the lithium acetate single-stranded carrier DNA/PEG method. Methods in Microbiology 26 53-66.
Peterson K R (2007) Preparation of intact yeast artificial chromosome DNA for transgenesis of mice. Nature Protocols 2 (11) 3009-3015.
Beverly S M (1988) Characterization of the ‘unusual’ mobility of large circular DNAs in pulsed field-gradient electrophoresis. Nucleic Acids Research 16(3) 925-939.
Kawasaki K, Minoshima S, Nakato E, Shibuya K, Shintani A, Asakawa S, Sasaki T, Klobeck H G, Combriato G, Zachau H G, Shimizu N. Evolutionary dynamics of the human immunoglobulin □ locus and the germline repertoire of the V□ genes. (2001) Eur J Immunol. 31, 1017-1028.
Wagner, S. D., Gross, G., Cook, G. P., Davies, S. L. and Neuberger, M. S. (1996) Antibody expression from the core region of the human IgH locus reconstructed in transgenic mice using bacteriophage P1 clones. Genomics, 35, 405-414.
Nelson, D. J. and Brownstein, B. H. (1994) YAC libararies, a user's guide. Freeman and Company NY
Gu H, Wilson D and Inselburg J (1992) Recovery of DNA from agarose gels using a modified Elutrap™. Journal of Biochemical and Biophysical Methods 24, 45-50.
Davies, N. P., Popov, A. V., Zou, X. and Brüggemann, M. (1996) Human antibody repertoires in transgenic mice: Manipulation and transfer of YACs. Antibody Engineering: A Practical Approach, eds. J. McCafferty, H. R. Hoogenboom and D. J. Chiswell, pp. 59-76, IRL, Oxford.
Yang X W, Model P and Heintz N (1997) Homologous recombination based modification in Esherichia coli and germline transmission in transgenic mice of a bacterial artificial chromosome. Nature Biotechnology 15, 859-865.
Sambrook, J. andf Russell, D. W. Molecular Cloning: a laboratory manual. Cold Spring Harbor, N.Y., 2001.
Reddy, S. T., Ge, X., Miklos, A. E., Hughes, R. A., Kang, S. H. Hoi, K. H., Chrysostomou, C., Hunicke-Smith, S. P., Iverson, B. L. and Tucker, P. W., 2010. Monoclonal antibodies isolated without screening by analyzing the variable-gene repertoire of plasma cells. Nature biotechnology 28(9):965-969
Gao, X., Yo, P., Keith, A., Ragan, T. J., and Harris, T. K., 2003. Thermodynamically balanced inside-out (TBIO) PCR-based gene synthesis: a novel method of primer design for high-fidelity assembly of longer gene sequences. Nucleic Acid Research 31, e143.
Tiller, T., Meffre, E., Yurasov, S., Tsuiji, M., Nussenzweig, M. C., and Wardemann, H., 2008. Efficient generation of monoclonal antibodies from single human B cells by single cell RT-PCR and expression vector cloning. J Immunol. Methods 329: 112-124
Kay, M. A., He, C-Y., Chen Z-Y., 2010. A robust system for production of minicircle DNA vectors. Nature biotechnology 28(12): 1287-1289
Persson, P., Engstrom, A., Rassmussen, L. K., Holmberg, E., and Frandsen, T. P., 2010. Development of mass spectrometry based techniques for the identification and determination of compositional variability in recombinant polyclonal antibody products. Anal. Chem 82: 7274-7282
Popov, A. V., Bützler, C., Frippiat, J.-P., Lefranc, M. P., and Brüggemann, M. (1996) Assembly and extension of yeast artificial chromosomes to build up a large locus. Gene, 177, 195-205
Popov, A. V., Zou, X., Xian, J., Nicholson, I. C. and Brüggemann, M. (1999) A human immunoglobulin □ locus is similarly well expressed in mice and humans. J. Exp. Med., 189, 1611-1619
Marchuk. D. and Collins, F. S. (1988) pYAC-RC, a yeast artificial chromosome vector for cloning DNA cut with infrequently cutting restriction endonucleases. Nucl. Acids Res., 16, 7743.
Ma, B., Pan, S. J., Zupancic, M. L. and Cormack, B. P. (2007) Assimilation of NAD⁺ precursors in Candida glabrata. Mol. Microbiol., 66, 14-25.
Gietz D and Woods R A (1998) Transformation of yeast by the lithium acetate single-stranded carrier DNA/PEG method. Methods in Microbiology 26 53-66.
Peterson K R (2007) Preparation of intact yeast artificial chromosome DNA for transgenesis of mice. Nature Protocols 2 (11) 3009-3015.
Beverly S M (1988) Characterization of the ‘unusual’ mobility of large circular DNAs in pulsed field-gradient electrophoresis. Nucleic Acids Research 16(3) 925-939.
Kawasaki K, Minoshima S, Nakato E, Shibuya K, Shintani A, Asakawa S, Sasaki T, Klobeck H G, Combriato G, Zachau H G, Shimizu N. Evolutionary dynamics of the human immunoglobulin □ locus and the germline repertoire of the V□ genes. (2001) Eur J Immunol. 31, 1017-1028.
Wagner, S. D., Gross, G., Cook, G. P., Davies, S. L. and Neuberger, M. S. (1996) Antibody expression from the core region of the human IgH locus reconstructed in transgenic mice using bacteriophage P1 clones. Genomics, 35, 405-414.
Nelson, D. J. and Brownstein, B. H. (1994) YAC libararies, a user's guide. Freeman and Company NY
Gu H, Wilson D and Inselburg J (1992) Recovery of DNA from agarose gels using a modified Elutrap™. Journal of Biochemical and Biophysical Methods 24, 45-50.
Davies, N. P., Popov, A. V., Zou, X. and Brüggemann, M. (1996) Human antibody repertoires in transgenic mice: Manipulation and transfer of YACs. Antibody Engineering: A Practical Approach, eds. J. McCafferty, H. R. Hoogenboom and D. J. Chiswell, pp. 59-76, IRL, Oxford.
Yang X W, Model P and Heintz N (1997) Homologous recombination based modification in Esherichia coli and germline transmission in transgenic mice of a bacterial artificial chromosome. Nature Biotechnology 15, 859-865.
Sambrook, J. and Russell, D. W. Molecular Cloning: a laboratory manual. Cold Spring Harbor, N.Y., 2001.
Andreason, G. L. and Glen Evans, G. A. Analytical Biochemistry, 180, 269, 1989
Baker, A. and Cotton, M. NAR 25, 1950, 1997
Transy C, Moingeon P, Stebbins C, and Reinherz E L (1989) Deletion of the cytoplasmic region of the CD3e subunit does not prevent assembly of a functional T-cell receptor. Proc. Natl. Acad. Sci. USA 86, 7108-7112

1. A bispecific three-chain antibody-like molecule (TCA) comprising
(a) an antibody heavy and light chain pair, or a functional fragment thereof, comprising at antigen-binding region specifically binding to a first binding target and at least one heavy chain constant region sequence; and
(b) a heavy chain antibody comprising an antigen-binding region specifically binding to a second binding target, and a CH2, CH3 and/or CH3 region, in the absence of a CH1 region.

2. The bispecific TCA of claim 1, wherein the antibody heavy and light chain pair comprises an antigen-binding region specifically binding to a first binding target and a CH1 sequence.

3. The bispecific TCA of claim 1, wherein the antibody heavy and light chain pair comprises an antigen-binding region specifically binding to a first binding target and a CH1 and a CH2 sequence.

4. The bispecific TCA of claim 1, wherein the antibody heavy and light chain pair comprises an antigen-binding region specifically binding to a first binding target and a CH1, a CH2, and a CH3 sequence.

5. The bispecific TCA of claim 1, 2, 3, or 4, wherein the antibody heavy and light chain pair further comprises a hinge region.

6. The bispecific TCA of claim 1, wherein the antibody heavy and light chain, or functional fragments thereof, are covalently linked to each other.

7. The bispecific TCA of claim 6, wherein the antibody heavy and light chain, or functional fragments thereof, are linked by a disulfide bond.

8. The bispecific TCA of claim 1, wherein the heavy chain antibody comprises an antigen-binding region specifically binding to a second binding target and a CH2 region, in the absence of a CH1 region.

9. The bispecific TCA of claim 8, wherein the heavy chain antibody comprises a CH3 region, in the absence of a CH1 region.

10. The bispecific TCA of claim 9, wherein the heavy chain antibody comprises a CH4 region, in the absence of a CH1 region.

11. The bispecific TCA of claim 8, 9 or 10, wherein the heavy chain antibody comprises a hinge region.

12. The bispecific TCA of claim 1, wherein the first and second binding targets are two different antigens.

13. The bispecific TCA of claim 12, wherein the first antigen is a cell surface antigen expressed by a target cell and the second antigen is expressed by an effector cell.

14. The bispecific TCA of claim 1, wherein the first and second binding targets are different epitopes of the same antigen.

15. The bispecific TCA of claim 1, wherein at least one of the first and second binding targets is part of a CD3 complex.

16. The bispecific TCA of claim 1, wherein at least one of the first and second binding targets is a CD3 antigen.

17. The bispecific TCA of claim 16, wherein the CD3 antigen is CD3 epsilon.

18. The bispecific TCA of claim 1 which is humanized or human.

19. A pharmaceutical composition comprising the bispecific TCA of claim 1, or any one of claims 12-18.

20. A kit comprising a container containing the bispecific TCA of claim 1 and instructions directing the user to utilize the composition.

21. A method for the production of the bispecific TCA of claim 1 comprising expressing the antibody heavy and light chain pair and the heavy chain antibody in a single host cell.

22. The method of claim 21 wherein the host cell is a prokaryotic cell.

23. The method of claim 21 wherein the host cell is an eukaryotic cell.

24. The method of claim 23 wherein the eukaryotic cell is a mammalian cell.

25. A method for the treatment of a cancer, comprising administering to a subject diagnosed with said cancer an effective amount of the bispecific TCA of claim 1.

26. The method of claim 25 wherein the cancer is selected from the group consisting of ovarian cancer, breast cancer, gastrointestinal, brain cancer, head and neck cancer, prostate cancer, colon cancer, lung cancer, leukemia, lymphoma, sarcoma, carcinoma, neural cell tumors, squamous cell carcinomas, germ cell tumors, metastases, undifferentiated tumors, seminomas, melanomas, myelomas, neuroblastomas, mixed cell tumors, and neoplasias caused by infectious agents.

27. A method for the treatment of an autoimmune disease or inflammatory condition comprising administering to a subject in need an effective amount of the bispecific TCA of claim 1.

28. A method for the treatment of an infectious disease caused by bacteria, viruses or parasites, comprising administering to a subject in need an effective amount of the bispecific TCA of claim 1.
